(19) United States
(12) Patent Application Publication
(10) Pub. No.: US 2006/0047214 A1
(43) Pub. Date: Mar. 2, 2006

(54) Wireless medical probe

(76) Inventor: Jacob Fraden, La Jolla, CA (US)

Correspondence Address: Jacob Fraden;Advanced Monitors Corporation, # 125, 6215 Ferris Sq., San Diego, CA, 92121, US

(21) Appl. No.: 10/924,155

(22) Filed: Aug. 24, 2004

(51) Int. Cl. A61B 5/04; A61B 5/02; A61B 5/00

(52) U.S. Cl. 600/513; 600/549; 600/485

(57) ABSTRACT

A wireless medical transducer that is attached to a patient's body contains one or more sensing assemblies for continuous, wireless and non-invasive monitoring of vital signs. These include EKG, core temperature, arterial blood pressure, arterial blood oxygenation, and others. A transducer may be configured either as a two-unit device where the units are connected by a short cable or a single unit. Sharing various components allows different vitals signs to be monitored with greater efficiency. Multiple radio transmitters may operate in the same environment without interfering with each other.

### FIELD OF INVENTION

The present invention relates generally to monitoring of vital signs of a patient, and more particularly to a system and method for monitoring one or more vital signs by means of a wireless communication. The invention is based on U.S. Provisional Patent Application No. 60/493,574 filed on Aug. 8, 2003.

### BACKGROUND OF INVENTION

Devices for measuring various physiological parameters, or “vital signs,” of a patient such as temperature, blood pressure, EKG, etc., have been a standard part of medical care for many years. Indeed, vital signs of some patients (e.g., those undergoing relatively moderate to high levels of care or being in a high risk category) typically are measured on a substantially continuous basis. This enables physicians, nurses and other health care providers to detect sudden changes in a patient's condition and evaluate a patient's condition over an extended period of time. Another important application of such devices is a home monitoring of a patient and alarming a care taker of critical changes in a vital sign status. And another possible applications is for the space exploration—continuous monitoring of the astronauts health while in a space vehicle or station. The similar type of a real time field monitoring can be envisioned for a military use when assessment of state of health and well-being of combat personnel may be a critical factor in military operations.

Since multiple vitals signs should be monitored simultaneously from a patient whose mobility should be limited to a lesser extent possible, it is highly desirable to devise a wireless system with maximum reliability and simplicity. Although a few “mobile” monitoring systems have been attempted, such systems are difficult to use and prone to failure resulting in the loss of a patient's vital signs data.

### DESCRIPTION OF PRIOR ART

Transmission of medical information is well known in art as a bio-telemetry. It may incorporate a one-way or two-way communication with a monitoring station as is exemplified by U.S. Pat. No. 6,577,893 issued to Besson et al. Numerous devices have been proposed for the wireless patient monitoring. Another example is a wireless temperature monitor according to U.S. Pat. No. 6,238,354 issued to Alvarez.

Most of devices for wireless transmission of data, as well as devices with wired connection, contain a sensing portion that is geared for monitoring just one and sometimes two vitals signs. The main issue with such sensing devices is incorporation of various sensors into a small package that is to be attached to the patient's body. Several separate sensors may interfere with one another and thus reduce usefulness of the device. Wireless EGK monitoring is known for nearly 60 years and is one of the easiest vital signs to monitor wirelessly. However, some vital sins detectors don't lend themselves to easy wireless monitoring due to either large size or inconvenient placement on the patient body or susceptibility to motion artifacts. For example, arterial blood pressure can be monitored either invasively with indwelled catheters or indirectly by applying an inflatable pressure cuff on an extremity. Neither method is acceptable for a convenient wireless monitoring of a moving patient. Another indirect method of blood pressure monitoring is analysis of a plethysmographic wave as describe in paper published by K. Meigas et al. (Continuous blood pressure monitoring using pulse wave delay. In: 2001. Proceedings of the 23^{rd} Ann. EMBS Intern. Conf., Istanbul, Turkey). Yet, the electrode arrangement proposed in the paper requires placement of four electrodes at four separate locations of a patient body which is quite inconvenient. Another example of a vital sign that could be monitored non-invasively is a deep body temperature as taught by U.S. Pat. No. 6,220,750 issued to Palti. While may be effective for a wired monitoring, that device incorporates a heater that requires a sizable power supply which is a serious limitation for a portable wireless device.
Thus, it is a goal of this invention to provide a small size vital signs probe that can be applied on a patient body;
Another goal of the invention is to provide a sensing arrangement that can monitor deep body temperature from a surface body with minimum energy requirement from multiple patients;
And another goal of this invention is to provide a combination electrode for EKG and electroplethysmographic signals that is suitable for a wireless communication;
It is a further goal of this invention to provide an system for non-invasive monitoring of indirect arterial blood pressure; and
And the final goal of this invention is to provide a simple reliable multi-channel wireless patient monitoring system.

### SUMMARY OF INVENTION

A combination non-invasive patient monitoring probe comprises one or more physiological transducers with signal conditioning circuits, power supply, data conversion and wireless transmission means. A combination of transducers where some components are shared for obtaining signals allows for simultaneous continuous monitoring of EKG, arterial blood oxygenation, deep body (core) temperature, arterial pressure and other vital signs.

### BRIEF DESCRIPTION OF DRAWINGS

FIG. 1 is a general view of a two-unit wireless monitoring system

FIG. 2 depicts a cross-sectional view of the probe

FIG. 3 is a cross-sectional view of the probe with pulse oximetry function

FIG. 4 is a bottom view of the electrodes

FIG. 5 shows a bottom view of the electrode and pulse oximetry components

FIG. 6 depicts a deep body temperature transducer

FIG. 6a shows a single-unit transducer attached to a patient body

FIG. 7 is a block-diagram of the wireless monitoring system

FIG. 8 shows time dependence of two temperature sensors

FIG. 9 depicts two variable components for the red and infrared portion of a spectrum

FIG. 10 shows a plethysmographic wave with different decaying slopes

FIG. 11 depicts a time delay between EKG and plethysmographic wave

FIG. 12 shows dependence between time delay and arterial pressure

FIG. 13 show two probe and two receivers operating on the same frequency

FIG. 14 is a timing diagram of transmitted codes

FIG. 15 shows attachment of adhesive cap to the transducer

### DESCRIPTION OF PREFERRED EMBODIMENT

Vital sign signals are collected non-invasively from a surface of the patient body 1 by a two-unit probe 2 as shown in FIG. 1. Probe 2 is a combination of first transducer 3, second transducer 4 and link 8 which may be a cable. Both transducers 3 and 4 contain various sensors, detectors, a power supply, and other components that will be described below in greater detail. Probe 2 is a self-containing device that collects, conditions and transmits information via communication link to receiver 7, which receives, processes and makes use of such information. The communication may be provided via a cable (wired), radio or optical (wireless) communication channel. As an illustration, FIG. 1 shows wireless radio signal 5 that enters receiving antenna 6 of receiver 7. Receiver 7 may contain some kind of an output device 125 such as a recorder, display or alarm. Push button 29 is used to initiate operation of probe 2 and for other functions that will be described below.

It should be noted that a two-unit probe 2 as shown in FIG. 1 is not the only possible configuration of the probe. For some applications, only a one-unit probe is needed (e.g. for temperature only monitoring) while for some vital signs, three or more units linked together may be required. A number of transducers should not construe a limitation of this invention.

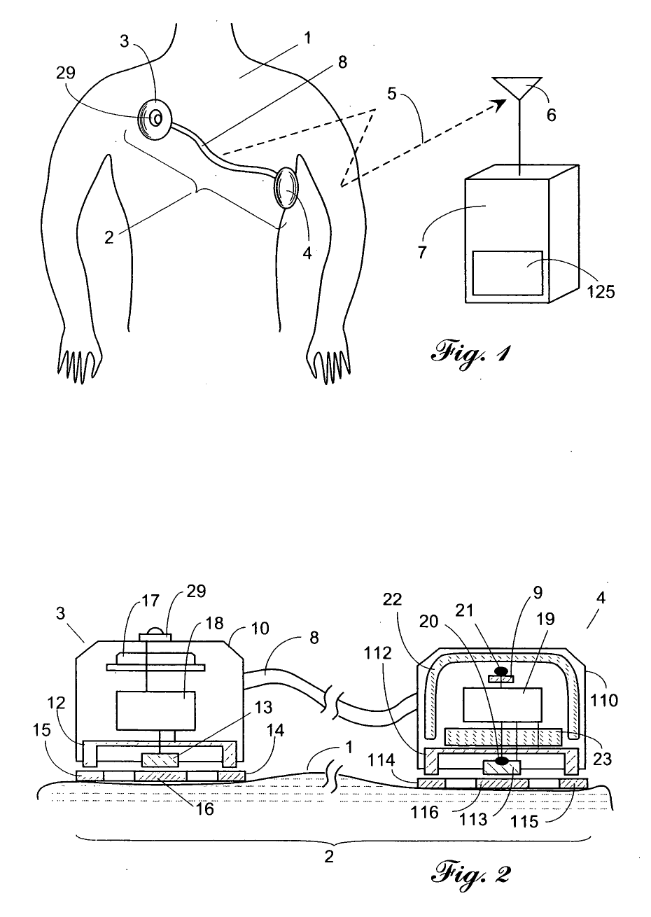

FIG. 2 illustrates a two-unit probe intended for simultaneous collecting of three types of a signal: EKG, electmoplethysmographic impedance (Z-value), and deep body (core) temperature (T). Any other combination of such is also possible, for example, EKG and Z-value, EKG and temperature, or temperature alone. It should be noted that EKG requires at least two separated electrodes to be attached to a patient body, Z-signal requires four electrodes: two for passing electric current and two for measuring a voltage drop. Temperature sensing requires one thermal contact attached to the patient body. FIG. 2 depicts the electrodes that share functions for receiving different vital signs and instead of seven contact areas on the body that would be required by the independent vital sign detectors, it has only four such areas within two sensing units.

Transducers 3 and 4 are housed respectively in first 10 and second 110 housings, and connected together by link 8. That link may provide electrical, optical or a combination of such connections. Bottom portions of housings 10 and 110 are placed on patient's skin 1. In this example, first transducer 3 contains power supply 17, push button 29, first electronic module 19, first EKG electrode 12 and first current electrode 13. Electrodes 12 and 13 are the electrophysiological electrodes that are intended for electrical interfaces with a human body, Thus, these electrodes my need to be fabricated of silver (or silver coated) plates with the outer AgCl coating as it is commonly done for such electrodes. To make an electrical contact with a human body, an electrically conductive gel pads may be also required. For practical use, these pads should have adhesive layers. First adhesive pad 14 contains first EKG pad 15 and first current pad 16. The adhesive portion is not shown in FIG. 2. It is important that pad 14 makes a good electrical contact between patient body 1 and electrodes 12 and 13. The silver-silver chloride electrodes and the interface gel pads are well known in art and are not described here in detail. FIG. 4 shows a bottom view of transducers 3 and 4 of FIG. 2.

To obtain both the EKG and Z-signal, another set of electrodes is required. This is provided by second transducer 4 which has the identical second EKG electrode 112, second current electrode 113 and the corresponding second adhesive pad 114 with second EKG pad 115 and second current pad 116. Here second current electrode 113 is somewhat different from first current electrode 13 because electrode 113 has attached to it first temperature sensor 20. Second current electrode 113 and first temperature sensor 20 must be in the intimate thermal contact. Further, second current pad 116 must be thin (about 0.001-0.005″) to minimize its thermal resistance and improve thermal coupling to patient body 1. Deep body (core) temperature of the patient can't be measure by first temperature sensor 20 alone because of influence of the ambient temperature. For computation of a deep body temperature, second transducer 4 is provided with second temperature sensor 21, outer insulator 20, and inner insulator 23. To improve stability of second temperature sensor 21, it can be attached to a metal plate 9.

All electrodes and temperature sensors are connected to the appropriate circuits inside the first and second electronic modules 18 and 19 respectively. The circuits get operating energy from power supply 17. One of the electronic modules incorporates a communication device which may be a radio transmitter.

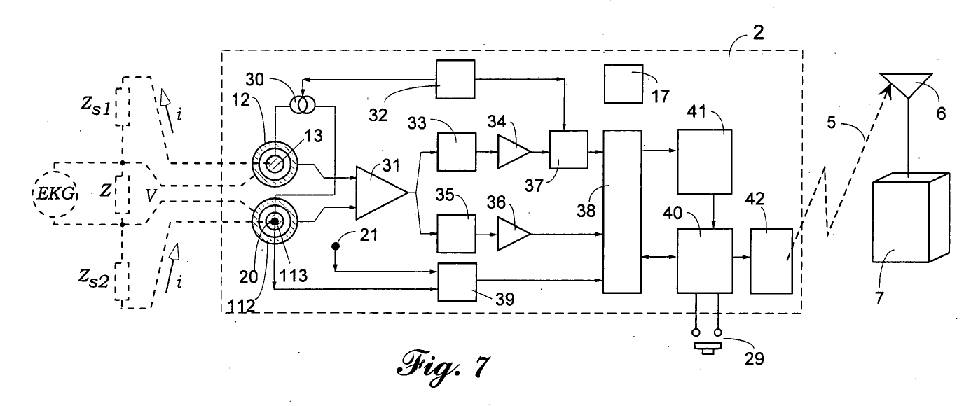

For the operational description of probe 2 refer to FIG. 7 which is a block diagram of a two-unit probe. On the left side of the diagram, there is an equivalent circuit of the patient body shown with dotted lines. Probe 2 of FIG. 7 receives and processes three vital signs: EKG, electroplethysmogram (EPG or Z-signal) and core temperature. Z-signal is a resistive component Z of the body internal electrical impedance. It depends on the body fluid content, cardiac output, peripheral vascular resistance and other variables. The EKG signal is generated by heart. Temperature is the result of cellular metabolism, the body physiological activity and other factors.

The circuit operates as follows. Oscillator 32 running at a typical frequency in the range from 10 kHz to 100 kHz controls a.c. current source 30 that forces current i into the patient's body through first and second current electrodes 13 and 113 respectively. Since the skin impedances Zₛ₁ and Zₛ₂ have strong capacitive components, most of the a.c. voltage drop develops over the internal resistive component Z. Voltage V is the sum of the a.c. voltage drop over resistance Z and the EKG voltage originated from the patient's heart. That combined voltage is picked-up by first and second EKG electrodes 12 and 112 respectively and passed to a broadband pre-amplifier 31. The output of the preamplifier is fed into two filters. The first one is high-pass filter 33 that allows a passing only of the frequencies corresponding to oscillator 32 and not of EKG. These frequencies are further amplified by first amplifier 34 and applied to synchronous demodulator 37 that is controlled by oscillator 32. The output low frequency signal from demodulator 37 represents value Z which is commonly called electroplethysmographic or reographic signal. It is fed into multiplexer 38 which is an analog gate. The low frequency components corresponding to the EKG signals pass from pre-amplifier 31 to low-pass filter 35, second amplifier 36 and subsequently to the same multiplexer 38. Thus, high frequency components of the spectrum originated in oscillator 32 are blocked out.

Signals from first and second temperature sensors 20 and 21 respectively are conditioned by temperature circuit 39 and also pass to multiplexer 38. Microcontroller 40 controls multiplexer 38, analog-to-digital (A/D) converter 41 and transmitter 42. The multiplexed signals in a digital format are transmitted to receiver 7 along with some other related information from probe 2, such as the probe identification (I.D) number, calibrating constants, etc. It should be noted that microcontroller 40 may incorporate memory that accumulates vital signs information for some time and then transmits it to receiver 7 in compact bundles on a periodic basis, say once every minute. This allows to minimize power consumption and reduce continuous transmission time.

To reduce power consumption, oscillator 32 my generate low duty-cycle pulses rather then continuous oscillation. This would force short current pulses through impedance Z and the average current supplied by the battery is greatly reduced. Alternatively, oscillator 32 may be controlled by the EKG signal from amplifier 36, thus measuring impedance only during the intervals that are required for data processing, for example, immediately after the R-wave of EKG.

In most applications, for example in a hospital room or while monitoring astronauts in flight, several radio-transmitting probes may need to operate in close proximity to one another. Even if the transmitted power is low, there is still a probability that the information may be picked up by the wrong receiver because all transmitters may operate within the same radio bandwidth. Besides reducing transmitting power, two other methods are used to prevent the cross-reception. One is a time division and the other is coding.

Time division works as follows. Each transmitter sends information is short packets with a low duty cycle. For example, a transmission may take 0.6 s with 1 minute intervals which is equivalent to duty cycle of 1%, meaning that there is only 1% probability that a signal from one transmitter will coincide with the signal from the second transmitter. The duty cycles may be made randomly variable, so that a probability of the respective overlapping becomes even smaller.

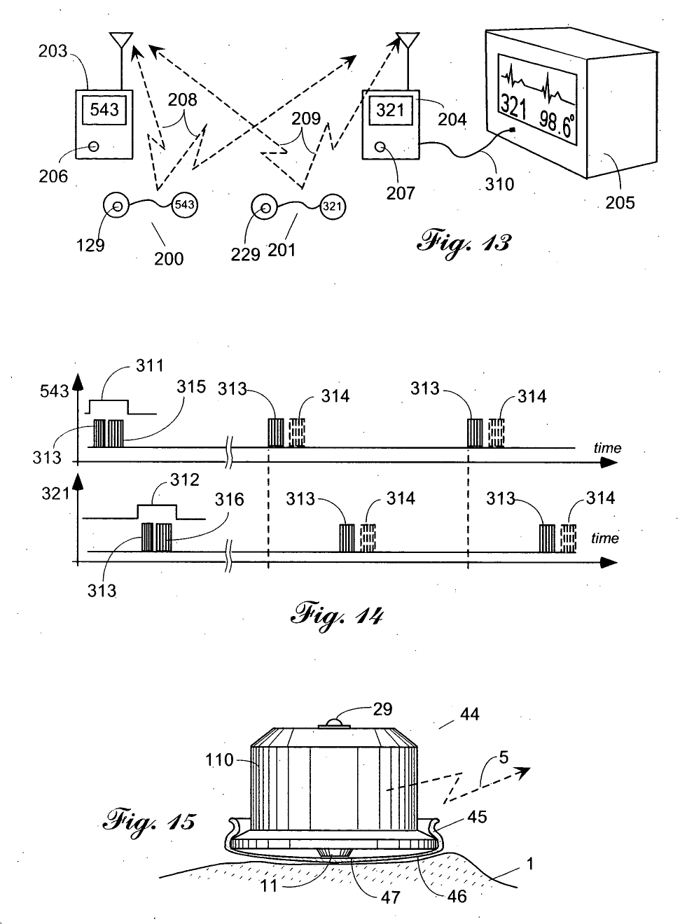

The coding method works as follows. Each transmitter is assigned at a factory a unique ID code. FIG. 13 illustrates two probes 200 and 201 operating within the same space and transmitting the corresponding radio signals. 208 and 209 on the same frequency which can be picked up by both receivers 203 and 204. As an illustration, the first receiver 203 is a self-containing device with a display and the second receiver 204 is an interface device between probe 201 and bedside monitor 205 which is connected to second receiver 204 by cable 310. Before operation, a set-up procedure for each pair (probe-receiver) is required. This can be accomplished by establishing the initial set-up communication, first between probe 200 and receiver 203 and then between second probe 201 and its receiver 204. Momentary switch 206 on receiver 203 is depressed which sets strobe 211 (see FIG. 14) inside that receiver making the receiver receptive to a set-up procedure. After that, push button 129 (the same as pushbutton 29 in FIGS. 1-3) on probe 200 is depressed. In response, probe 200 transmits its unique ID code 313 and the set-up code 315. In this example, transmitter 200 has the ID code “543”. Receiver 203 receives the code and sets itself to be receptive only to data that carry that particular code. Note that since switch 207 on second receiver 204 was not depressed at that particular time, receiver 204 ignores the set up procedure for probe 200. However, receiver 204 is coded in a similar manner by using switch 207 and pushbutton 229 on second probe 201. In a similar manner, this sets receiver 204 to be receptive only to probe 201 that has a unique ID code (“321” in the example). From that moment on, probes 200 and 201 go to operation mode and transmit medical information codes 314 accompanied by their unique ID codes 313. The coding forces each receiver to accept only information codes 314 from the corresponding probe and ignore other transmissions that have different ID codes.

To preserve energy contents of power supply 17 in probe 2 (FIG. 7) while not in use, signals from first and second temperature sensors 20 and 21 respectively are compared with each other and if they indicate a very small temperature gradient, say less than 0.5 degree C. for a prolonged period of time of all hour or more, this will indicate that probe 2 is no linger attached to a patient. Another possible way to detect disconnection from a patient is monitoring of current i. If this current drops to zero, a patient is no longer connected. In this cases, power of probe 2 can be automatically shut down by microcontroller 40. It can be restored by depressing pushbutton 29.

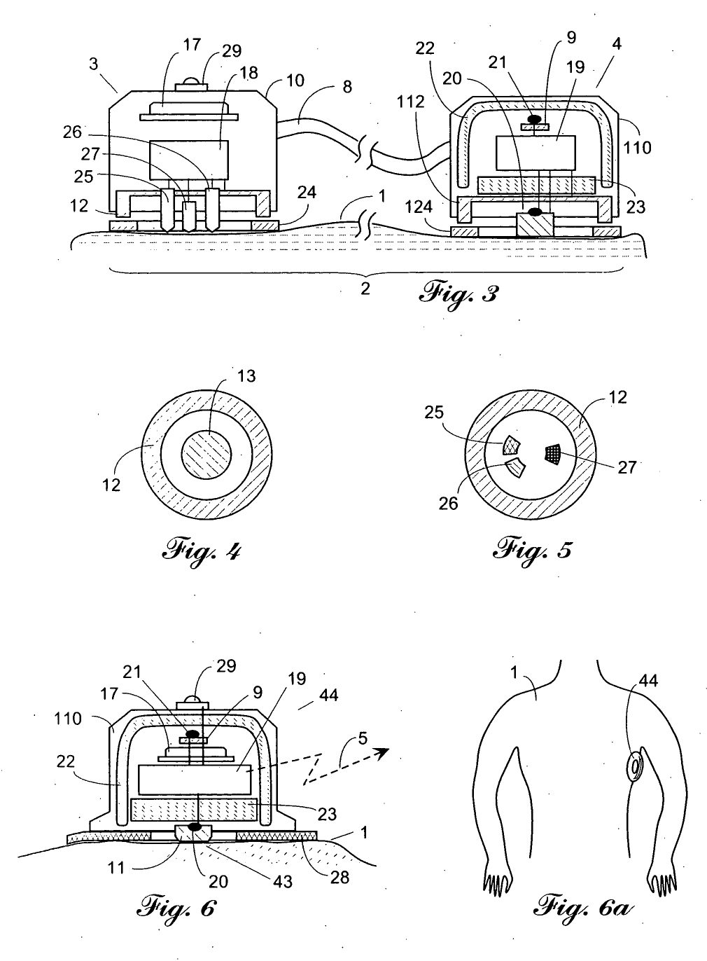

Another possible configuration of probe 2 is shown in FIG. 3. Instead of the Z-value (EPG), it detects two photo-plethysmographic (PPG) signals at two different light wavelengths, say in red and infrared (IR). First transducer 3 now contains the optical components: first LED 25 (red), second LED 26 (IR) and light detector 27. Detecting photoplethysmogram at these two wavelengths allows computation of the arterial blood oxygenation which is known in art as pulse oximetry. The optical components as identified above are positioned adjacent to the EKG electrode, for example, inside of a circular EKG electrode 12 as shown in FIG. 5. The pulsating components which are modulated by light passing to and reflecting from the patient's body are measured and transmitted to the receiver. The detected red and IR signals, 104 and 103 respectively, have different magnitudes as shown in FIG. 9. The ratio of these magnitudes is commonly used to compute the degree of oxygen saturation of hemoglobin, SpO₂, in arterial blood. We do not describe this process further as such computation is well known in art of patient monitoring

Since receiver 7 receives the EKG and either EPG or PPG signals, these two signals can be used to compute the arterial blood pressure by using one of the following methods. In the first method, only either EPG or PPG is analyzed. The decaying (back) slope of the detected EPG or PPG wave (FIG. 10) correlates with the peripheral vascular resistance of the circulatory system and, subsequently, with the mean arterial blood pressure. The slower decaying slope 107 indicates higher mean arterial blood pressure, the faster decaying slope 108 is an indication of a lower pressure, whereas a medium slope 106 indicates a normal blood pressure. Another way of computing the mean blood pressure is to measure time delay between the rapid portions of EKG and the EPG or PPG waves as shown in FIG. 11. Time delay Δt of the EPG (PPG) can be measured with respect to either Q or R waves of the EKG. Two thresholds 212 and 213 cross the EKG and EPG (PPG) waves at the corresponding points 214 and 215, allowing measurement of Δt. FIG. 12 illustrates dependence of mean arterial pressure 220 of time delay Δt. The systolic pressure 222 and diastolic pressure 221 can be estimated from the extreme corresponding points S and D on the PPG or EPG wave (see FIG. 11) by a proportional scaling. Naturally, these methods require an individual patient calibration against one of the conventional blood pressure measurements. The measurements as indicated above can be performed by microcontroller 40 or, preferably, inside receiver 7.

As it was indicated above, depending on the application, probe 2 may be configured in multiple ways. One common application is a deep body temperature sensing. A single-unit temperature probe is shown in FIG. 6 as transducer 44. In many respects it is identical to transducers 4 in FIGS. 2 and 3, except that it contains no electrodes, because now its purpose is only the temperature monitoring. Second housing 110 contains outer and inner insulators 22 and 23 respectively, first and second temperature sensors 20 and 21, second electronic module 19 and power supply 17. The probe may be attached to patient's body 1 by a double-sided adhesive disk 28 (see also FIG. 6a). In the lower center of transducer 44, there is metal contact 11 attached to first temperature sensor 20. Temperature sensors may be thermistors, semiconductors or, alter natively, one of them may be a thermocouple junction, while the other such junction must be thermally attached to another temperature sensor.

FIG. 15 shows an alternative way of attaching transducer 44 to the patient's body 1. Here cap 45 has an adhesive bottom 46. The cap is snapped onto transducer 44 and holds it on patient's 1 skin. Lower portion 47 of cap 45 is thin (on the order of 0.001″) so that its thermal conductivity is rather high, much higher than that of patient's skin. The cap may be fabricated by a thermo-forming process from polypropylene or any other suitable material.

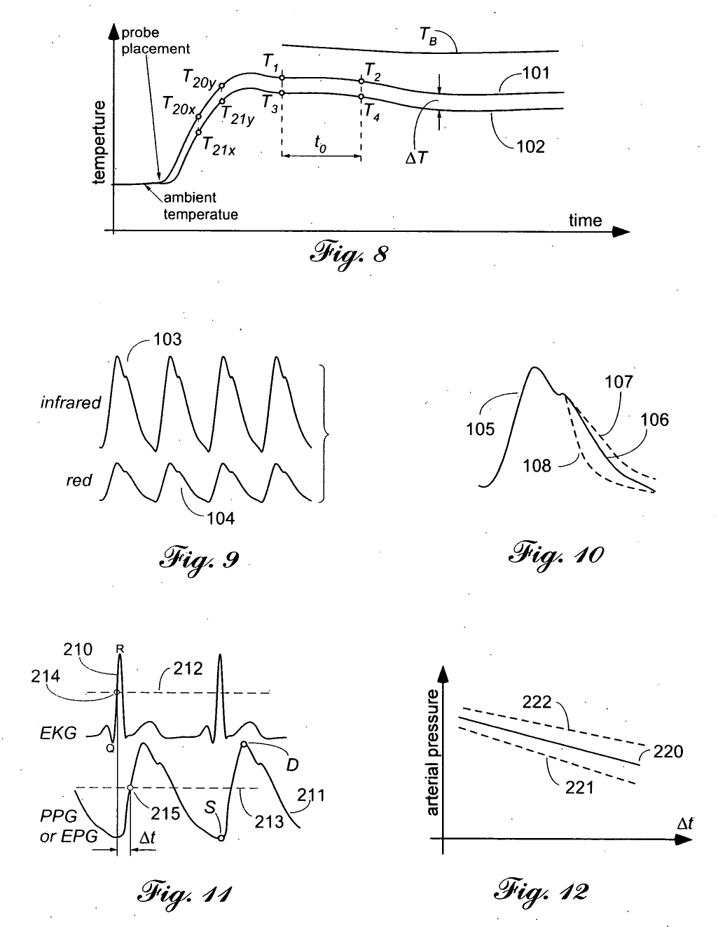

A deep body temperature is measured as follows. Since first temperature sensor 20 is in an intimate thermal contact with the patient body (FIG. 6), it measures temperature of patient's skin 43 which commonly is cooler than the core temperature. Second temperature sensor 21 is removed from first temperature sensor 20 and insulated from it by inner insulator 23. Thus, second temperature sensor 21 measures the interior temperature of the transducer. Insulators 22 and 23 may be just the air gaps near the corresponding temperature sensors. Plate 9 attached to that sensor helps to improve its thermal stability. FIG. 8 shows time changes of temperature 101 measured by first temperature sensor 20 and temperature 102 measured by second temperature sensor 21. After the probe placement on the patient body, both temperatures increase above ambient, though there is a thermal gradient ΔT=T₁₀₁−T₁₀₂ between them. This thermal gradient is a measure of the heat flow from a deep body interior to the first and subsequently to the second temperature sensors 20 and 21. On the basis of the Newton's law of cooling, the deep body temperature may be computed from temperatures 101 and 102 as
T_{B}=T₁₀₁+μΔT (1)
where μ is the experimentally calibrated factor, typically ranging from 1.5 to 3. It should be noted that its value may also depend on both T₁₀₁ and T₁₀₂, so for a higher accuracy a more complex function needs to be employed to compute core temperature. An example of such a function is
T_{B}=AT₁₀₁²+(B+CT₁₀₂)T₁₀₁+DT₁₀₂+B (2)
where A, B, C, D and E are the experimentally determined constants.

While the above description contains many specifics, these specifics should not be construed as limitations on the scope of the invention, but merely as exemplifications of preferred embodiments thereof. Those skilled in the art will envision many other possible variations that are within the scope and spirit of the invention.

1. A medical monitor for collecting, transmitting and receiving vital signs from surface of a patient body contains in combination
a first probe housing;
a first bottom portion of the first probe housing that contacts patient body;
a first sensor of a vital sign positioned adjacent to said first bottom portion;
a first electronic module positioned internally to first probe housing;
transmitter of electromagnetic radiation positioned internally to first probe housing;
a power supply positioned internally to first probe housing;
receiver of electromagnetic radiation that is detached from the first probe housing;
output device connected to said receiver.

2. A medical monitor of claim 1 further comprising a second probe housing attached to patient body and containing
a second sensor of a vital sign
second electronic module
a link for connecting to said first electronic module.
relating arterial blood pressure to said time delay

3. A medical monitor of claim 1, where
a first sensor is a first temperature sensor that is thermally insulated from said outer portion of the probe housing;
said probe housing further comprising a second temperature sensor positioned inside said probe housing and thermally insulated from first temperature sensor

4. A method of computing arterial pressure of a patient comprising steps of
obtaining EKG signal
obtaining plethysmographic signal
transmitting EKG and plethysmographic signals to a processing means;
measuring time delay between a rapid wave of the EKG signal and rapid slope of the plethysmographic signal
relating the measured time delay to patient's arterial pressure

5. A method of computing arterial pressure of a patient comprising steps of
obtaining plethysmographic signal;
transmitting plethysmographic signals to a processing means;
measuring rate of a decaying slope of a plethysmographic signal;
relating said rate to patient's arterial pressure
